SEQUENCE LISTING (21 sequences)

SEQ ID NO: 1 - DNA, 480 bp, Artificial Sequence
  source: /note=“Description of Artificial Sequence Synthetic polynucleotide”
  modified_base (100)..(100): a, c, t, g, unknown or other
  modified_base (140)..(140): a, c, t, g, unknown or other
  modified_base (258)..(258): a, c, t, g, unknown or other
  modified_base (424)..(424): a, c, t, g, unknown or other
  modified_base (437)..(437): a, c, t, g, unknown or other
  modified_base (479)..(479): a, c, t, g, unknown or other
ggtcctgtctgtggtgacccagtgtgactctcatggggagcaaggagaggaagttgaagttcactgacagggttgttaaggggattatgcaatagatganacccatgggcctgaagtccgagggtgtatgttagttcccngttcttttgacccatggattaacctactctgtgcaaagggcattttcaagtttgttgccctgctcacttggagaaagcttatgaaggatcaggaaaattaaaagggtgctctcgcctntaacttctctctcctttgctttcacaggccttggtatgttcctgcttcatsccyttctacagtggccttatccctccttccttcagaggcgtggtaagtcggctttctctgctagcgctgagtcctgggggcctctgaagtgtgctcacacatctcctgcctgcanggcactggtgtcnggcacctcagggtctgtcccatggtggagccccatgcctcant

SEQ ID NO: 2 - DNA, 20 bp, Artificial Sequence
  source: /note=“Description of Artificial Sequence Synthetic primer”
ttgctttcacaggccttggt

SEQ ID NO: 3 - DNA, 22 bp, Artificial Sequence
  source: /note=“Description of Artificial Sequence Synthetic primer”
ggagggataaggccactgtaga

SEQ ID NO: 4 - DNA, 15 bp, Artificial Sequence
  source: /note=“Description of Artificial Sequence Synthetic probe”
ttcctgcttcatscc

SEQ ID NO: 5 - DNA, 13 bp, Artificial Sequence
  source: /note=“Description of Artificial Sequence Synthetic probe”
cctgcttcatscc

SEQ ID NO: 6 - DNA, 23 bp, Artificial Sequence
  source: /note=“Description of Artificial Sequence Synthetic primer”
cctctgaaggaaggagggataag

SEQ ID NO: 7 - DNA, 25 bp, Artificial Sequence
  source: /note=“Description of Artificial Sequence Synthetic primer”
aacttctctctcctttgctttcaca

SEQ ID NO: 8 - DNA, 22 bp, Artificial Sequence
  source: /note=“Description of Artificial Sequence Synthetic primer”
ggagggataaggccactgtaga

SEQ ID NO: 9 - DNA, 22 bp, Artificial Sequence
  source: /note=“Description of Artificial Sequence Synthetic primer”
gaaggagggataaggccactgt

SEQ ID NO: 10 - DNA, 26 bp, Artificial Sequence
  source: /note=“Description of Artificial Sequence Synthetic primer”
aacttctctctcctttgctttcacag

SEQ ID NO: 11 - DNA, 15 bp, Artificial Sequence
  source: /note=“Description of Artificial Sequence Synthetic probe”
gttcctgcttcatsc

SEQ ID NO: 12 - DNA, 14 bp, Artificial Sequence
  source: /note=“Description of Artificial Sequence Synthetic probe”
ttcctgcttcatsc

SEQ ID NO: 13 - DNA, 1092 bp, Artificial Sequence
  source: /note=“Description of Artificial Sequence Synthetic polynucleotide”
tttttttttttttttttttttttttttttttttattatactctaagttttagggtacatgtgcacattgtgcaggttagttacatatgtatacatgtgccatgctggtgcgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaattgcggtgtttggttttttgttcttgcgatagtttactgagaatgatggtttccaatttcatccatgtccctacaaaggatatgaactcatcattttttatggctgcatagtattccatggtgtatatgtgccacattttcctaatccagtctatcattgttggacatttgggttggttccaagtctttgctattgtgaatagtgccgcaataaacatacgtgtgcatgtgtctttatagcagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcaaaaattttctcccatgttgtaggttgcctgttcactctgatggtagtttcttttgctgtgcagaagctctttagtttaattagatcccatttgtcaattttgtcttttgttgccattgcttttggtgttttggacatgaagtcctt

SEQ ID NO: 14 - DNA, 19 bp, Artificial Sequence
  source: /note=“Description of Artificial Sequence Synthetic primer”
gcctgaagtccgagggtgt

SEQ ID NO: 15 - DNA, 20 bp, Artificial Sequence
  source: /note=“Description of Artificial Sequence Synthetic primer”
tcagaggcccccaggactca

SEQ ID NO: 16 - DNA, 21 bp, Artificial Sequence
  source: /note=“Description of Artificial Sequence Synthetic primer”
tgttgccctgctcacttggag

SEQ ID NO: 17 - DNA, 15 bp, Artificial Sequence
  source: /note=“Description of Artificial Sequence Synthetic probe”
ttcctgcttcatgcc

SEQ ID NO: 18 - DNA, 13 bp, Artificial Sequence
  source: /note=“Description of Artificial Sequence Synthetic probe”
cctgcttcatccc

SEQ ID NO: 19 - DNA, 15 bp, Artificial Sequence
  source: /note=“Description of Artificial Sequence Synthetic probe”
gttcctgcttcatgc

SEQ ID NO: 20 - DNA, 14 bp, Artificial Sequence
  source: /note=“Description of Artificial Sequence Synthetic probe”
ttcctgcttcatcc

SEQ ID NO: 21 - DNA, 1093 bp, Artificial Sequence
  source: /note=“Description of Artificial Sequence Synthetic polynucleotide”
aaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaggaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcaattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaaaaaaaaaaaaaaaaaaaaaaaaa

(19) United States
(12) Patent Application Publication
(10) Pub. No.: US 2022/0017963 A1
(43) Pub. Date: Jan. 20, 2022

(54) Methods, Compositions and Systems for Detecting PNPLA3 Allelic Variants

(71) Applicant: Laboratory Corporation of America Holdings, Burlington, NC (US)

(72) Inventors: Shanshan Zhou, Cary, NC (US); Jeffrey Shuster, Burlington, NC (US)

(21) Appl. No.: 17/368,610

(22) Filed: Jul. 6, 2021

Related U.S. Application Data
(60) Provisional application No. 63/048,449, filed on Jul. 6, 2020.

(51) Int. Cl. C12Q 1/6883

(52) U.S. Cl. CPC C12Q 1/6883; C12Q 2600/156

(57) ABSTRACT

Disclosed herein are methods and kits for detection of a single nucleotide polymorphism (SNP) rs738409 of a Patatin-like phospholipase domain containing 3 (PNPLA3) gene in a subject. Such methods are useful in the diagnosis of a subject with NASH and the selection of a particular treatment for the subject. In some embodiments, the provided methods allow for a highly selective detection of SNP rs738409.

### RELATED APPLICATIONS

This application claims priority to U.S. Provisional Application No. 63/048,449 filed Jul. 6, 2020. The disclosure of U.S. Provisional Application No. 63/048,449 is incorporated by reference in its entirety herein.

### SEQUENCE LISTING

The instant application contains a Sequence Listing which has been submitted electronically in ASCII format and is hereby incorporated by reference in its entirety. Said ASCII copy, created on Sep. 16, 2021, is named 057618-1257138_SL.txt and is 9,430 bytes in size.

### FIELD OF THE INVENTION

This disclosure relates to methods, compositions and systems for detecting variants in the PNPLA3 gene.

### BACKGROUND

The patatin-like phospholipase domain-containing protein 3 (PNPLA3) gene encodes adiponutrin, which is a triacylglycerol lipase responsible for mediating triacylglycerol hydrolysis in adipocytes. PNPLA3 is located on the forward strand of the long arm of chromosome 22 (GRCH38 coordinate of 43,923,739-43,964,488) which encodes a human lipase that is distributed between hepatic lipid droplets and cell membranes. PNPLA3 I148M (rs738409, GRCh38.p7 chr22:43928847 C>G) is a germline mutation strongly associated with the development of Non-alcoholic fatty liver diseases (NAFLDs), including nonalcoholic steatohepatitis (NASH). Non-synonymous substitution of C to G corresponds to the missense amino acid substitution of Methionine for Isoleucine. The risk allele frequency of rs738409 in the general global population is ˜27%. (Younossi et. al., 2018 Nat. Rev. Gastro. Hepatol. 15(1):11-20).

PNPLA3 rs738409 C>G reflects one of the critical genetic factors that confers high-risk to NASH. NASH is a progressive and devastating disease with no approved therapies to date. NASH is currently the most common liver disease worldwide, and 5% of the U.S. population are projected to have NASH by 2033 (United States Census Bureau). NASH develops over a long period of time and is influenced by both environmental and genetic factors.

Currently available methods for genotyping detecting the C>G variant of PNPLA3 SNP rs738409 can be expensive, time consuming, and/or lack the necessary specificity and selectivity. Several SNP genotyping methods involving hybridization, ligation, or DNA polymerases are known in the art, including Sanger sequencing, allele-specific polymerase chain reaction (PCR), and oligonucleotide ligation assay.

Genotyping tests are routinely designed using publically available gene information in public databases and accessed, for example, using ensembl (Howe et al., Ensembl 2021, Nuc. Acids Res, 49: D884-D891). If there are genome variants in persons for whom the test will be employed that are not reported in these databases, the tests that are designed based on the publically available information may either not be successful at all, or significantly, may yield incorrect genotyping results. Thus, there is a need for new highly specific and selective genotypic assays.

### SUMMARY

Embodiments of the present disclosure comprise compositions, methods, and systems for the diagnosis and treatment of liver disease such as a non-alcoholic fatty liver disease and related syndromes. The present disclosure provides, among other things, a novel method for detecting a single nucleotide polymorphism (SNP) rs738409 of a Patatin-like phospholipase domain containing 3 (PNPLA3) gene in a subject in the presence or absence of a genetic variant upstream or downstream of the PNPLA3 gene region. In some embodiments, the method is capable of detecting the SNP rs738409 in the presence or absence of a previously unreported genetic insertion upstream of the SNP rs738409 in human populations. In particular, the novel method provided herein can be used to assist in the clinical diagnosis of NASH or other nonalcoholic fatty liver diseases (NAFLDs), risk of progression of disease, and to assist in the identification of patients who may benefit from a particular treatment. The present disclosure may be embodied in a variety of ways.

In a first aspect of the present disclosure is a method for detecting the single nucleotide polymorphism (SNP) rs738409 of a Patatin-like phospholipase domain containing 3 (PNPLA3) gene in a subject comprising: providing at least one oligonucleotide probe, wherein the probe comprises a nucleic acid specific for an allele of the SNP rs738409. The method may further comprise providing a set of oligonucleotide PCR primers, wherein the primers are PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409. In an embodiment, amplification is performed using quantitative polymerase chain reaction (qPCR) amplification. The method may further comprise the step of contacting a sample comprising a nucleic acid from the subject with at least one probe such that hybridization occurs between the probe and the nucleic acid from the subject and detecting binding of the probe to the SNP rs738409 by qPCR using the rs738409-specific primers. The method may also comprise determining the genotype of the subject at the SNP rs738409 loci as homozygous for C, homozygous for G, or a heterozygote (C/G).

In a second aspect, the present disclosure provides a composition and/or kit comprising at least one primer probe/set for detecting a SNP rs738409 of a PNPLA3 gene in a subject. In certain embodiments, the primer/probe set comprises: (a) at least one oligonucleotide probe, wherein the probe comprises a nucleic acid specific for an allele of the SNP rs738409; and (b) a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409. In an embodiment, the probes are labeled with a detectable moiety.

For example, in some embodiments, the method may comprise (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote.

Also disclosed are systems for performing any of the steps of the disclosed methods, and/or for using any of the disclosed compositions and/or kits.

### BRIEF DESCRIPTION OF THE FIGURES

The present disclosure may be better understood by referring to the following non-limiting figures.

FIG. 1 shows the DNA sequence flanking the focal SNP rs738409 with example positions for standard PCR primers for Sanger sequencing. SNP rs738409 is located 3 bp upstream of SNP rs738408. The rs738409 SNP is located at position 43928847. SNP rs738408 is located at position 43928850. The position of a novel insertion variant is shown by the arrow. The bold text depicts one exon of the PNPLA3 gene. The non-bold text depicts portions of the intron regions.

FIG. 2 depicts a custom assay design (primer/probe set 1) of a PNPLA3 I148M genotyping assay in accordance with embodiments of the disclosure. The forward primer is 20 bp long, starting at position 94. The reverse primer is 22 bp long, starting at position 155. Probe 1 is specific for the G allele of SNP rs738409. Probe 1 is 15 bp long, starting at position 117. Probe 2 is specific for the C allele of rs738409. Probe 2 is 13 bp long, starting at position 119. Position 1 corresponds to Chr22:43928717.

FIG. 3 depicts a custom assay design (primer/probe set 2) of a PNPLA3 I148M genotyping assay in accordance with embodiments of the disclosure. The forward primer is 20 bp long, starting at position 94. The reverse primer is 23 bp long, starting at position 167. Probe 1 is specific for the G allele of SNP rs738409. Probe 1 is 15 bp long, starting at position 117. Probe 2 is specific for the C allele of rs738409. Probe 2 is 13 bp long, starting at position 119. Position 1 corresponds to Chr22:43928717.

FIG. 4 depicts a custom assay design (primer/probe set 3) of a PNPLA3 I148M genotyping assay in accordance with embodiments of the disclosure. The forward primer is 25 bp long, starting at position 80. The reverse primer is 22 bp long, starting at position 155. Probe 1 is specific for the G allele of SNP rs738409. Probe 1 is 15 bp long, starting at position 117. Probe 2 is specific for the C allele of rs738409. Probe 2 is 13 bp long, starting at position 119. Position 1 corresponds to Chr22:43928717.

FIG. 5 depicts a custom assay design (primer/probe set 4 of a PNPLA3 I148M genotyping assay in accordance with embodiments of the disclosure. The forward primer is 25 bp long, starting at position 80. The reverse primer is 22 bp long, starting at position 158. Probe 1 is specific for the G allele of SNP rs738409. Probe 1 is 15 bp long, starting at position 117. Probe 2 is specific for the C allele of rs738409. Probe 2 is 13 bp long, starting at position 119. Position 1 corresponds to Chr22:43928717.

FIG. 6 depicts a custom assay design (primer/probe set 5) of a PNPLA3 I148M genotyping assay in accordance with embodiments of the disclosure. The forward primer is 26 bp long, starting at position 80. The reverse primer is 22 bp long, starting at position 158. Probe 1 is specific for the G allele of SNP rs738409. Probe 1 is 15 bp long, starting at position 116. Probe 2 is specific for the C allele of rs738409. Probe 2 is 14 bp long, starting at position 117.

FIG. 7 shows a process map summarizing a genotyping method protocol in accordance with embodiments of the disclosure.

FIG. 8 shows an exemplary computing device in accordance with various embodiments of the disclosure.

FIG. 9 depicts a PNPLA3 rs738409 Insert Sequence (+ strand) (SEQ ID NO:13).

FIG. 10 depicts a PNPLA3 rs738409 Insert Sequence (− strand) (SEQ ID NO:21).

### DETAILED DESCRIPTION OF THE INVENTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples.

The term “active fragment” is a portion of a primer that can be used for amplification of the rs738409 SNP.

The term “allele” refers to different versions of a nucleotide sequence of a same genetic locus (e.g., a gene). In a diploid organism with two sets of chromosomes, there is one of a gene (allele) on each chromosome.

The term “allelic discrimination plot” refers to the plot of normalized reporter signal from a first allele probe plotted against the normalized reporter signal from a second allele probe.

The term “amplification” refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100 fold difference). Amplification by asymmetric PCR is generally linear. A skilled artisan will understand that different amplification methods may be used together.

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “baseline” refers to a cycle-to-cycle range that defines background fluorescence in the amplification plot. The term “biological sample” encompasses any sample obtained from a biological source. A biological sample can, by way of non-limiting example, include cell-free DNA, blood, serum, plasma, amniotic fluid, sera, urine, feces, epidermal sample, skin sample, cheek swab, sperm, amniotic fluid, cultured cells, bone marrow sample and/or chorionic villi. In some embodiments, the sample may be a dried biological sample, such as but not limited to dried blood or plasma. Convenient biological samples may be obtained by, for example, scraping cells from the surface of the buccal cavity. The term biological sample encompasses samples which have been processed to release or otherwise make available a nucleic acid or protein for detection as described herein. For example, a biological sample may include a cDNA that has been obtained by reverse transcription of RNA from cells in a biological sample. The biological sample may be obtained from a stage of life such as a fetus, young adult, adult, and the like. Fixed or frozen tissues also may be used.

The term “coding sequence” refers to a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. In an embodiment, the coding sequence is on the (+) strand of genomic DNA. The anti-sense strand (i.e., the (−) strand) is the complement of such a nucleic acid, and the encoding sequence can be deduced therefrom. As used herein, the term “non-coding sequence” refers to a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

The terms “complement,” “complementary” and “complementarity,” refer to the pairing of nucleotide sequences according to Watson/Crick pairing rules. For example, a sequence 5′-GCGGTCCCA-3′ has the complementary sequence of 5′-TGGGACCGC-3′. A complement sequence can also be a sequence of RNA complementary to the DNA sequence. Certain bases not commonly found in natural nucleic acids may be included in the complementary nucleic acids including, but not limited to, inosine, 7-deazaguanine, Locked Nucleic Acids (LNA), and Peptide Nucleic Acids (PNA). Complementary need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

The term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

The term “deletion” encompasses a mutation that removes one or more nucleotides from a naturally-occurring nucleic acid.

The terms “delta Rn” or “dRn” or “baseline-corrected normalized reporter” refer to the difference in normalized fluorescence signal generated by the reporter between the pre-PCR read and the post-PCR read.

The term “end-point data” refers to data collected at the end of the PCR process.

The terms “flanking” or “flanks” mean that a primer hybridizes to a target nucleic acid adjoining a region of interest sought to be amplified on the target. The skilled artisan will understand that preferred primers are pairs of primers that hybridize 3′ from a region of interest, one on each strand of a target double stranded DNA molecule, such that nucleotides may be add to the 3′ end of the primer by a suitable DNA polymerase. For example, primers that flank mutant PNPLA3 sequence do not actually anneal to the mutant sequence but rather anneal to sequence that adjoins the mutant sequence. In some cases, primers that flank a PNPLA3 exon are generally designed not to anneal to the exon sequence but rather to anneal to sequence that adjoins the exon (e.g. intron sequence). However, in some cases, amplification primer may be designed to anneal to the exon sequence.

The term “genotype” refers to the genetic constitution of an organism. More specifically, the term refers to the identity of alleles present in an individual. “Genotyping” of an individual or a DNA sample refers to identifying the nature, in terms of nucleotide base, of the two alleles possessed by an individual at a known polymorphic site.

As used herein, “genotypic data” are data about the genotype of, for example, a virus. Examples of genotypic data include, but are not limited to, the nucleotide or amino acid sequence of a virus, a part of a virus, a viral gene, a part of a viral gene, or the identity of one or more nucleotides or amino acid residues in a viral nucleic acid or protein.

The term “heterozygous” or “HET” refers to an individual possessing two different alleles of the same gene. As used herein, the term “heterozygous” encompasses “compound heterozygous” or “compound heterozygous mutant.” As used herein, the term “compound heterozygous” refers to an individual possessing two different alleles. As used herein, the term “compound heterozygous mutant” refers to an individual possessing two different copies of an allele, such alleles are characterized as mutant forms of a gene. The term “mutant” as used herein refers to a mutated, or potentially non-functional form of a gene.

The term “homozygous” refers to an individual possessing two copies of the same allele. As used herein, the term “homozygous mutant” refers to an individual possessing two copies of the same allele, such allele being characterized as the mutant form of a gene. The term “mutant” as used herein refers to a mutated, or potentially nonfunctional form of a gene.

The term “hybridize” or “hybridization” refers to a process where two complementary nucleic acid strands anneal to each other under appropriately stringent conditions. Oligonucleotides or probes suitable for hybridizations typically contain 10-100 nucleotides in length (e.g., 18-50, 12-70, 10-30, 10-24, 18-36 nucleotides in length). Nucleic acid hybridization techniques are well known in the art. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

The term “insertion” or “addition” refers to a change in an amino acid or nucleotide sequence resulting in the addition of one or more amino acid residues or nucleotides, respectively, as compared to the naturally occurring molecule.

The term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

The terms “labeled” and “labeled with a detectable” agent or moiety and “labeled with a visually detectable” agent or moiety are used herein interchangeably to specify that an entity (e.g., a nucleic acid probe, antibody, etc.) can be visualized, for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents (described in more detail herein) include, but are not limited to, radionucleotides, fluorophores, chemiluminescent agents, microparticles, enzymes, colorimetric labels, magnetic labels, haptens, molecular beacons, aptamer beacons, and the like.

The term “linked” or “genetically linked” refers to s the tendency of DNA sequences that are close together on a chromosome to be inherited together during meiosis, as two genetic markers that are physically near to each other are unlikely to be separated onto different chromatids during chromosomal crossover and are therefore said to be more “linked” than markers that are far apart.

The terms “minor groove binder” or “MGB” or “MGB moiety” refer to a molecule that selectively binds to the minor groove of DNA, a shallow furrow in the DNA helix. When conjugated to the 3′ end of an oligonucleotide, an MGB moiety can function as a non-extendable blocker moiety.

The term “multiplex PCR” refers to amplification of two or more regions which are each primed using a distinct primers pair.

The terms “no template control” or “NTC” refers to a negative control sample used in qPCR amplification reactions that tests for possible reagent contamination (e.g., in the master mix).

The terms “normalized reporter” or “Rn” refer to fluorescence signal from the reporter dye normalized to the fluorescence signal of the passive reference dye.

The term “genotypic assay” is a test that determines a genetic sequence of an organism, a part of an organism, a gene or a part of a gene.

The term “primer” refers to a short single-stranded oligonucleotide capable of hybridizing to a complementary sequence in a nucleic acid sample. Typically, a primer serves as an initiation point for template dependent DNA synthesis. Deoxyribonucleotides can be added to a primer by a DNA polymerase. In some embodiments, such deoxyribonucleotides addition to a primer is also known as primer extension. The term primer, as used herein, includes all forms of primers that may be synthesized including peptide nucleic acid primers, locked nucleic acid primers, phosphorothioate modified primers, labeled primers, and the like. A “primer pair” or “primer set” for a PCR reaction typically refers to a set of primers typically including a “forward primer” and a “reverse primer.” As used herein, a “forward primer” refers to a primer that anneals to the anti-sense strand of dsDNA. A “reverse primer” anneals to the sense-strand of dsDNA.

The term “polymorphism” refers to the coexistence of more than one form of a gene or portion thereof or a non-coding nucleic acid sequence.

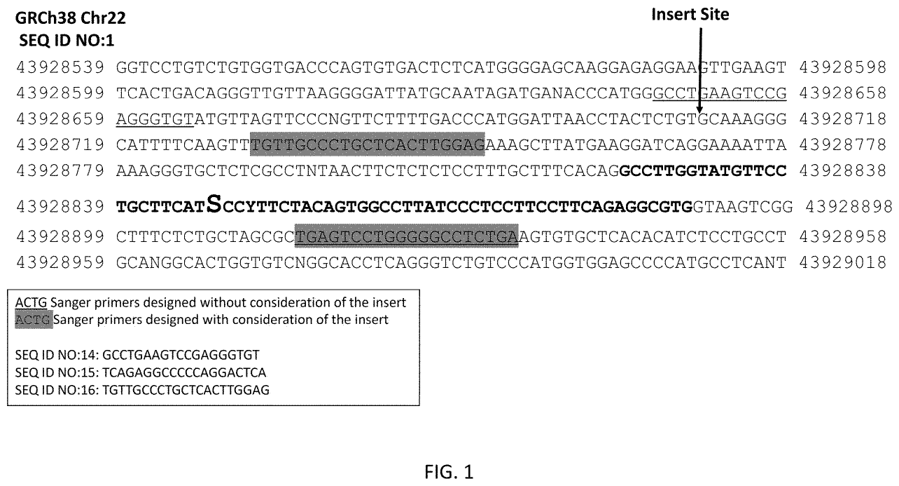

The term “position” refers to the nucleotide numbering in the PNPLA3 gene sequence as illustrated in FIG. 1 where position 1 corresponds to Chr22:43928717.

The term “real-time PCR” refers to the use of quantitative polymerase chain reaction (qPCR) to amplify DNA across several orders of magnitude, generating thousands to millions of copies of a particular DNA sequence in a manner such that amplification is measured in real-time. In certain embodiments, qPCR or real-time PCR, employs the use of a probe, which binds to the target sequence prior to amplification. As amplification proceeds, the probe is released. In an embodiment, release of the probe is measured by separation of a quencher moiety and a fluorescent moiety, such that measuring fluorescence may be used to quantify the amplification process.

The term PNPLA3-specific PCR primers refers to primers that amplify the region of the human PNPLA3 gene that includes the region containing SNP rs738409 but not any upstream or downstream variants.

The term “sense strand” or (+) strand refers to the strand of double-stranded DNA (dsDNA) that includes at least a portion of a coding sequence of a functional protein. As used herein, the term “anti-sense strand” or (−) strand refers to the strand of dsDNA that is the reverse complement of the sense strand.

The term “specific,” when used in connection with an oligonucleotide primer, refers to an oligonucleotide or primer, under appropriate hybridization or washing conditions, is capable of hybridizing to the target of interest and not substantially hybridizing to nucleic acids which are not of interest. Higher levels of sequence identity are preferred and include at least 60%, 65%, 70%, 75%, 80%, 85%, 90%, 95%, 98%, 99%, or 100% sequence identity. In some embodiments, a specific oligonucleotide or primer contains at least 4, 6, 8, 10, 12, 14, 16, 18, 20, 22, 24, 26, 28, 30, 35, 40, 45, 50, 55, 60, 65, 70, or more bases of sequence identity with a portion of the nucleic acid to be hybridized or amplified when the oligonucleotide and the nucleic acid are aligned.

The term “subject” refers to a human or any non-human animal. A subject can be a patient, which refers to a human presenting to a medical provider for diagnosis or treatment of a disease. A human includes pre- and post-natal forms.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substitution” refers to the replacement of one or more amino acids or nucleotides by different amino acids or nucleotides, respectively, as compared to the naturally occurring molecule.

The term “trace score” refers to an average of basecall quality values for bases in the clear range in Sanger Sequencing.

The term “threshold” refers to the numerical value assigned for each run, which reflects a statistically significant point above the calculated baseline.

The term “wild-type” refers to the typical or the most common form existed in nature. For example, a wild-type PNPLA3 gene or protein refers to the typical or the most common form of PNPLA3 gene or protein existed in a natural population. As used herein, “wild-type” is used interchangeably with “naturally-occurring.”

The term “% sequence homology” is used interchangeably herein with the terms “% homology,” “% sequence identity” and “% identity” and refers to the level of amino acid sequence identity between two or more peptide sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 60, 70, 80, 85, 90, 95, 98% or more sequence identity to a given sequence

Unless noted otherwise, the standard one letter and three letter abbreviations for amino acids are used. When polypeptide sequences are presented as a series of one-letter and/or three-letter abbreviations, the sequences are presented in the N C direction, in accordance with common practice. Also, where specified, individual amino acids in a sequence are represented herein as AN, wherein A is the standard one letter symbol for the amino acid in the sequence, and N is the position in the sequence. Mutations are represented herein as A₁NA₂, wherein A₁ is the standard one letter symbol for the amino acid in the reference protein sequence, A₂ is the standard one letter symbol for the amino acid in the mutated protein sequence, and N is the position in the amino acid sequence. For example, a I148M mutation represents a change from isoleucine to methionine at amino acid position 148. Mutations may also be represented herein as NA₂, wherein N is the position in the amino acid sequence and A₂ is the standard one letter symbol for the amino acid in the mutated protein sequence (e.g., 148M, for a change from the wild-type amino acid to methionine at amino acid position 148). Additionally, mutations may also be represented herein as A₁N, wherein A₁ is the standard one letter symbol for the amino acid in the reference protein sequence and N is the position in the amino acid sequence (e.g., I148 represents a change from isoleucine to any amino acid at amino acid position 48). This notation is typically used when the amino acid in the mutated protein sequence is either not known or, if the amino acid in the mutated protein sequence could be any amino acid, except that found in the reference protein sequence. The amino acid positions are numbered based on the full-length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one-letter abbreviations, and the top strand of double-stranded sequences, are presented in the 5′→3′ direction.

Genotypic Detection of PNPLA3 SNP rs738409

In some embodiments, the PNPLA3 gene may comprise an insertion in the intron immediately upstream of exon 3. In certain embodiments, the insertion is a repetitive genomic sequence that is may have multiple copies in the human genome. In an embodiment, there may only be a single copy of this sequence on chromosome 22. The presence of this insertion sequence can, in some embodiments, confound detection of the rs738409 SNP. Thus, the disclosed methods, compositions and systems may be used to detect the rs738409 SNP in the presence or absence of the insertion. For example, in certain embodiments the insertion sequence may be positioned as indicated in FIG. 1 and has the sequence of SEQ ID NO: 13 (+) strand (SEQ ID NO:21 (−) strand).

In some embodiments, the methods, compositions, and systems of the present disclosure may be used for detection of a single germline mutation of SNP rs738409, located in exon 3 of the PNPLA3 gene. A portion of the sequence of PNPLA3 (SEQ ID NO:1), showing SNP rs738409 and the nearby polymorphism SNP rs738408, 3 bases downstream of SNP rs738408, and the position of an insertion variant is shown in FIG. 1. The target SNP (rs738409, C/G) is represented by “S” and the adjacent SNP (rs738408, T/C) is represented by “Y” in FIG. 1. Exemplary positions for standard PCR primers for Sanger sequencing are identified.

In certain instances, the methods, compositions (e.g., kits), and systems are capable of detecting the C>G mutation (rs738409) in the PNPLA3 gene. The PNPLA3 gene is located on the long arm of chromosome 22 at band 13.31 and is 40,750 bases in length. A mutation of isoleucine to methionine (I[ATC]>M[ATG]) as SNP rs738409 on exon 3 of the PNPLA3 gene has been shown to be associated with non-alcoholic fatty liver disease, including non-alcoholic steatosis (NASH). When compared to the C/C genotype, both the G/C and G/G genotypes are associated with NASH. In some embodiments, the assay results may be used to identify NASH patients with homozygous G/G (M/M) or heterozygous C/G (I/M) mutations who may benefit from a particular treatment. In some embodiments, the methods, compositions, kits, and systems are used to determine that a nucleic acid sample comprises the PNPLA3 I148M mutation by detecting a G allele at SNP rs738409.

Each of the embodiments of the methods, compositions, kits, and systems of the present disclosure can allow for the detection of the rs738409 SNP on the PNPLA3 gene in the presence or absence of any portion of a variant in the human PNPLA3 gene.

### Samples

In some embodiments, the methods, compositions, kits, and systems described herein are used for detection of a single nucleotide polymorphism (SNP) rs738409 of PNPLA3 gene in a subject. In some instances, a biological sample is taken from a subject. In some embodiments, the subject is a human. In further embodiments, the human subject has been diagnosed with a liver disease. In some embodiments, the liver disease is a non-alcoholic liver disease. In some embodiments the liver disease is NASH. In other embodiments, the human subject has not been diagnosed with a liver disease (e.g., NASH).

A biological sample may encompass any sample obtained from a biological source. A biological sample can, by way of non-limiting example, include cell-free DNA, serum, plasma, whole blood, amniotic fluid, sera, urine, feces, epidermal sample, skin sample, saliva, cheek swab, sperm, amniotic fluid, cultured cells, bone marrow sample and/or chorionic Convenient biological samples may be obtained by, for example, scraping cells from the surface of the buccal cavity. The biological sample may be obtained from a stage of life such as a fetus, young adult, adult, and the like. Dried samples (e.g., dried blood or plasma) or fixed or frozen tissues also may be used.

In some embodiments, the sample is whole blood. In certain instances, the sample comprises an anticoagulant. For example, in some embodiments, the sample is stored in a K₂EDTA tube. EDTA functions as an anticoagulant by preventing clotting by chelating calcium. In some embodiments, the biological sample is a fresh whole blood sample. For example, a fresh blood sample may be less than 5, 4, 3, 2, or 1 days within blood draw and stored at 4° C. In other embodiments, the biological sample is a frozen whole blood sample. For example, blood samples may be stored under −80° C. prior to use with the methods, compositions (e.g., kits), and systems described herein. In some instances, frozen biological samples undergo at least one freeze-thaw cycle. In some embodiments, frozen biological samples undergo multiple freeze-thaw cycles.

In certain embodiments, the biological sample comprises a nucleic acid. Nucleic acid analyses can be performed on cell-free DNA and/or genomic DNA, messenger RNA, and/or cDNA. In some instances, a nucleic acid is extracted from the biological sample. For example, in some embodiments, primers are designed to anneal to the exon and so mRNA (or cDNA made by reverse transcription of mRNA) may be used. In some embodiments, primers may anneal at least in part to the intron region(s) surrounding the exon; in these cases genomic DNA may be used as the template. The term biological sample encompasses samples that have been processed to release or otherwise make available a nucleic acid or protein for detection as described herein. In certain embodiments, the nucleic acid is genomic DNA. For example, genomic DNA can be extracted from any of, but not limited to, the following sources: serum, plasma, whole blood (e.g., whole blood in EDTA, ACD-A, ACD-B), saliva, cheek swabs, blood spots, amniotic fluid, chorionic villus samples (CVS) (for single exon sequencing only), and cultured cells (e.g., CVS, amniotic fluid, fibroblasts, POC). In further embodiments, extracted genomic DNA may be purified by any method generally known in the art. In some embodiments, the nucleic acid is quantified prior to use with the genotyping assay methods described herein.

### Probe and Primer Hybridization

In a first aspect of the present disclosure, described herein is a method for detection of a single nucleotide polymorphism (SNP) rs738409 of a Patatin-like phospholipase domain containing 3 (PNPLA3) gene in a subject comprising the steps of: (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote.

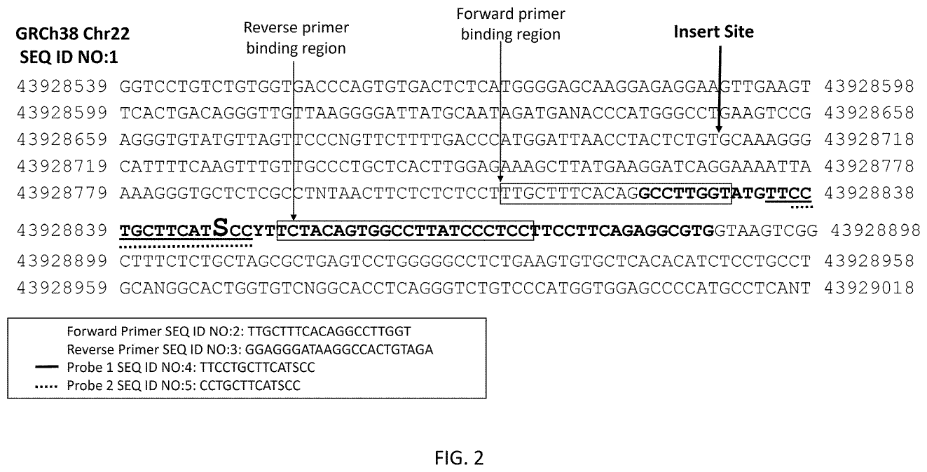

In some embodiments, the method comprises nucleic acid hybridization between a probe and a target nucleic acid sequence for a subject. In some embodiments, nucleic acids are analyzed by hybridization using one or more oligonucleotide probes specific for the rs738409 region in the PNPLA3 gene. In some embodiments, the probes comprise the nucleic acid sequence SEQ ID NO:4, SEQ ID NO:5, SEQ ID NO:11, SEQ ID NO:12, or SEQ ID NOS 17-20. Exemplary probes are shown in FIGS. 2-6 and described in more detail below.

Nucleic acid probes may comprise ribonucleic acids and/or deoxyribonucleic acids. In certain embodiments, the probes comprise deoxyribonucleic acid (DNA). In some embodiments, provided nucleic acid probes are DNA oligonucleotides (i.e., “oligonucleotide probes”). Generally, oligonucleotide probes are long enough to bind specifically to a homologous region of the PNPLA3 gene, but short enough such that a difference of one nucleotide between the probe and the nucleic acid sample being tested disrupts hybridization. Typically, the sizes of oligonucleotide probes vary from approximately 5 to 100 nucleotides. In some embodiments, oligonucleotide probes vary from 5 to 90, 5 to 80, 5 to 70, 5 to 60, 5 to 50, 5 to 40, 5 to 35, 5 to 30, 5 to 25, 5 to 20, 5 to 20 or 13 to 17 nucleotides in length. As appreciated by those of ordinary skill in the art, the optimal length of an oligonucleotide probe may depend on the particular methods and/or conditions in which the oligonucleotide probe may be employed.

Probes of the present disclosure include those that are capable of specifically hybridizing a mutant PNPLA3 allele containing a C>G mutation at the rs738409 loci. Probes of the present disclosure also include those that are capable of specifically hybridizing a normal allele in a particular region of the PNPLA3 gene and therefore capable of distinguishing a normal allele from a mutant PNPLA3 allele containing a C>G mutation at the rs738409 loci. For example, one of ordinary skill in the art could use probes described herein to determine whether an individual is homozygous or heterozygous for a particular allele.

Thus, in some embodiments, the probe is a mutant probe. A mutant probe is specific for the rs738409 G allele. In certain instances, the mutant probe described herein is capable of specifically hybridizing to the complement of the DNA sequence encoding a mutant allele (G) in the rs738409 loci of the PNPLA3 gene. In some embodiments, the mutant probe has a sequence of TTCCTGCTTCATgCC (SEQ ID NO:17) where the lower case letter indicates the allele of interest. In other embodiments, the mutant probe has a sequence of GTTCCTGCTTCATgC (SEQ ID NO:19) where the lower case letter indicates the allele of interest.

In other embodiments, the probe is a wild-type probe. A wild-type probe is specific for the rs738409 C allele. Thus, in some embodiments, the wild-type probe described herein is capable of specifically hybridizing to the complement of the DNA sequence encoding a normal allele (C) in the rs738409 loci of the PNPLA3 gene. In some embodiments, the wild-type probe has a sequence of CCTGCTTCATcCC (SEQ ID NO:18). In other embodiments, the wild-type probe has a sequence of TTCCTGCTTCATcC (SEQ ID NO:20) where the lower case letter indicates the allele of interest.

In some embodiments, the mutant and wild-type probes are designed such that the basic melting temperature (Tm) is approximately equal for both the mutant and wild-type probe. The Tm of the probe affects the ability of the probe to bind and amplify equally. In certain embodiments, the length of the probe is adjusted so that the mutant and wild-type probes have approximately the same Tm.

In certain embodiments, the probe comprise a minor groove binder (MGB) moiety.

Thus in certain embodiments, the method further comprises providing at least one oligonucleotide probe, wherein the probe comprises a minor groove binder (MGB) moiety, and a nucleic acid specific for an allele of the SNP rs738409. In some embodiments, the MGB probe incorporates a 5′ fluorescent reporter, a 3′ non-fluorescent quencher (NFQ) and a 3′ MGB moiety. MGB moieties are capable of associating with the minor groove of the target nucleic acid. A minor groove forms on the target nucleic acid following hybridization of an oligonucleotide probe and a specific target nucleic acid. In some embodiments, the probe comprises an MGB moiety at the 3′ terminus. In other embodiments, the probe comprises an MGB moiety at the 5′ terminus. In some embodiments, the probe is a TaqMan MGB probe, MGB moieties at the are capable of binding to the DNA helix minor groove. The interaction between the MGB moiety and the minor groove of the DNA can stabilize the MGB probe-template complex. Additionally, the MGB moiety can increase the melting temperature (Tm) of the probe to further stabilize probe binding and/or allow for shortening the probe length. In some embodiments, the probes are less than 20, 19, 18, 17, 16, 15, 14, or 13 bases. Shorter probes are generally beneficial for binding stability and improved allelic discrimination as the variable allele contributes more to the strength of hybridization.

For the specificity of the assay, the positions of the primers are designed to bind to the PNPLA3 gene distal to the position of the insertion variant (FIG. 1).

Various non-limiting embodiments of the probes and primers are disclosed in FIGS. 2-6. In FIGS. 2-6, the target SNP (rs738409, C/G) is represented by “S” and the adjacent SNP (rs738408, T/C) is represented by “Y”. The positions as presented in FIGS. 2-6 are correlated to the genomic numbering as provide in TABLE 1, where position 1 corresponds to Chr22:43928717.

TABLE 1
Position Correlation to Genomic Numbering

| | Forward Primer | Reverse Primer | VIC-Probe | FAM-Probe |
|---|---|---|---|---|
| | Coordinate | coordinate | Coordinate (G) | Coordinate (C) |
| Primer/Probe | Chr22: 43928812- | Chr22: 43928873- | Chr22: 43928835- | Chr22: 43928837- |
| Set 1 | 43928831 | 43928852 | 43928849 | 43928849 |
| Primer/Probe | Chr22: 43928812- | Chr22: 43928885- | Chr22: 43928835- | Chr22: 43928837- |
| Set 2 | 43928831 | 43928861 | 43928849 | 43928849 |
| Primer/Probe | Chr22: 43928798- | Chr22: 43928885- | Chr22: 43928835- | Chr22: 43928837- |
| Set 3 | 42928822 | 43928861 | 43928849 | 43928849 |
| Primer/Probe | Chr22: 43928798- | Chr22: 43928888- | Chr22: 43928835- | Chr22: 43928837- |
| Set 4 | 42928822 | 43928864 | 43928849 | 43928849 |
| Primer/Probe | Chr22: 43928798- | Chr22: 43928888- | Chr22: 43928834- | Chr22: 43928835- |
| Set 5 | 42928822 | 43928864 | 43928848 | 43928848 |

FIG. 2 depicts a custom assay design (primer/probe set 1) of a PNPLA3 I148M genotyping assay in accordance with embodiments of the disclosure. The length and position of the primers and probes is provided in TABLE 2. The forward primer is 20 bp long and starts at position 94. The reverse primer is 22 bp long and starts at position 155. Probe 1 is specific for the G allele of SNP rs738409. Probe 1 is 15 bp long and starts at position 117. Probe 2 is specific for the C allele of rs738409. Probe 2 is 13 bp long and starts at position 119.

TABLE 2
Primer/probe set 1

| Fwd | Fwd | Fwd | Fwd | Rev | Rev | Rev | Rev |
|---|---|---|---|---|---|---|---|
| Start | Length | Tm | % GC | Start | Length | Tm | % GC |
| 94 | 20 | 59 | 50 | 155 | 22 | 58 | 55 |

| Probe1 | Probe1 | Probe1 | Probe1 | Probe2 | Probe2 | Probe2 | Probe2 | Amp | |
|---|---|---|---|---|---|---|---|---|---|
| Start | Length | Tm | % GC | Start | Length | Tm | % GC | Len | Penalty |
| 117 | 15 | 66 | 53 | 119 | 13 | 67 | 62 | 62 | 62 |

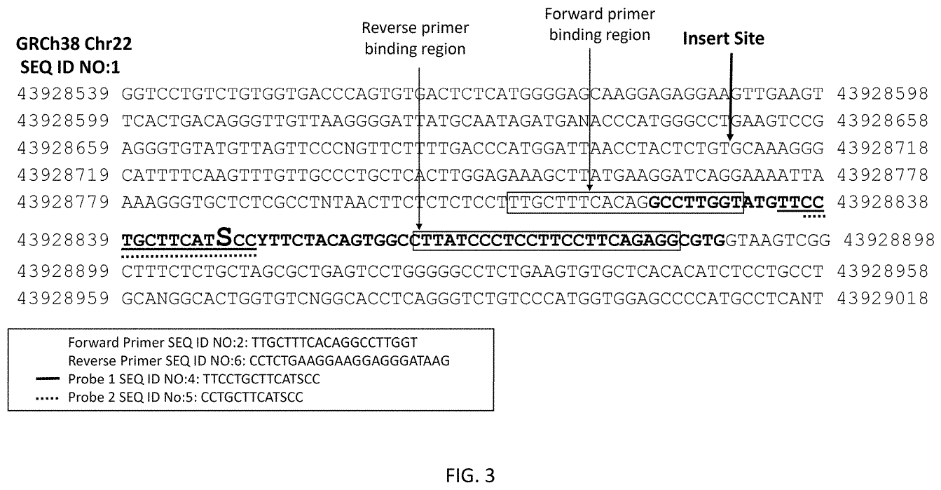

FIG. 3 depicts a custom assay design (primer/probe set 2) of a PNPLA3 I148M genotyping assay in accordance with embodiments of the disclosure. The length and position of the primers and probes is provided in TABLE 3. The length and position of the primers and probes is provided in TABLE 3. The forward primer is 20 bp long, starting at position 94. The reverse primer is 23 bp long, starting at position 167. Probe 1 is specific for the G allele of SNP rs738409. Probe 1 is 15 bp long, starting at position 117. Probe 2 is specific for the C allele of rs738409. Probe 2 is 13 bp long, starting at position 119.

TABLE 3
Primer/probe set 2

| Fwd | Fwd | Fwd | Fwd | Rev | Rev | Rev | Rev |
|---|---|---|---|---|---|---|---|
| Start | Length | Tm | % GC | Start | Length | Tm | % GC |
| 94 | 20 | 59 | 50 | 167 | 23 | 58 | 52 |

| Probe1 | Probe1 | Probe1 | Probe1 | Probe2 | Probe2 | Probe2 | Probe2 | Amp | |
|---|---|---|---|---|---|---|---|---|---|
| Start | Length | Tm | % GC | Start | Length | Tm | % GC | Len | Penalty |
| 117 | 15 | 66 | 53 | 119 | 13 | 67 | 62 | 74 | 123 |

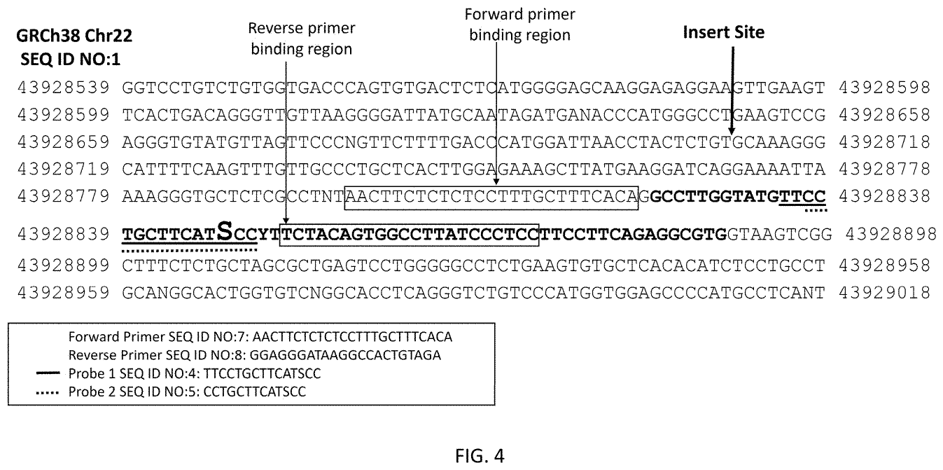

FIG. 4 depicts a custom assay design (primer/probe set 3) of a PNPLA3 I148M genotyping assay in accordance with embodiments of the disclosure. The length and position of the primers and probes is provided in TABLE 4. The forward primer is 25 bp long, starting at position 80. The reverse primer is 22 bp long, starting at position 155. Probe 1 is specific for the G allele of SNP rs738409. Probe 1 is 15 bp long, starting at position 117. Probe 2 is specific for the C allele of rs738409. Probe 2 is 13 bp long, starting at position 119.

TABLE 4
Primer/probe set 3

| Fwd | Fwd | Fwd | Fwd | Rev | Rev | Rev | Rev |
|---|---|---|---|---|---|---|---|
| Start | Length | Tm | % GC | Start | Length | Tm | % GC |
| 80 | 25 | 59 | 40 | 155 | 22 | 58 | 55 |

| Probe1 | Probe1 | Probe1 | Probe1 | Probe2 | Probe2 | Probe2 | Probe2 | Amp | |
|---|---|---|---|---|---|---|---|---|---|
| Start | Length | Tm | % GC | Start | Length | Tm | % GC | Len | Penalty |
| 117 | 15 | 66 | 53 | 119 | 13 | 67 | 62 | 76 | 137 |

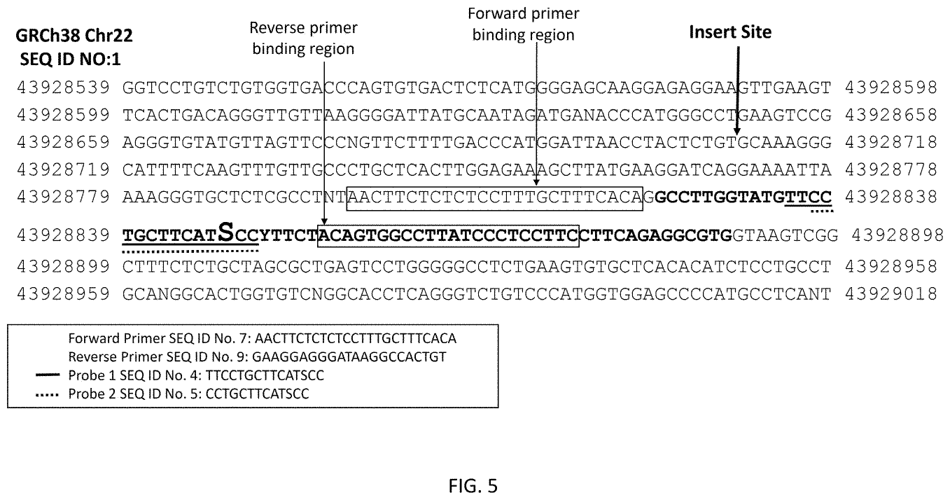

FIG. 5 depicts a custom assay design (primer/probe set 3) of a PNPLA3 I148M genotyping assay in accordance with embodiments of the disclosure. The length and position of the primers and probes is provided in TABLE 5. The forward primer is 25 bp long, starting at position 80. The reverse primer is 22 bp long, starting at position 158. Probe 1 is specific for the G allele of SNP rs738409. Probe 1 is 15 bp long, starting at position 117. Probe 2 is specific for the C allele of rs738409. Probe 2 is 13 bp long, starting at position 119.

TABLE 5
Primer/probe set 4

| Fwd | Fwd | Fwd | Fwd | Rev | Rev | Rev | Rev |
|---|---|---|---|---|---|---|---|
| Start | Length | Tm | % GC | Start | Length | Tm | % GC |
| 80 | 25 | 59 | 40 | 158 | 22 | 59 | 55 |

| Probe1 | Probe1 | Probe1 | Probe1 | Probe2 | Probe2 | Probe2 | Probe2 | Amp | |
|---|---|---|---|---|---|---|---|---|---|
| Start | Length | Tm | % GC | Start | Length | Tm | % GC | Len | Penalty |
| 117 | 15 | 66 | 53 | 119 | 13 | 67 | 62 | 79 | 152 |

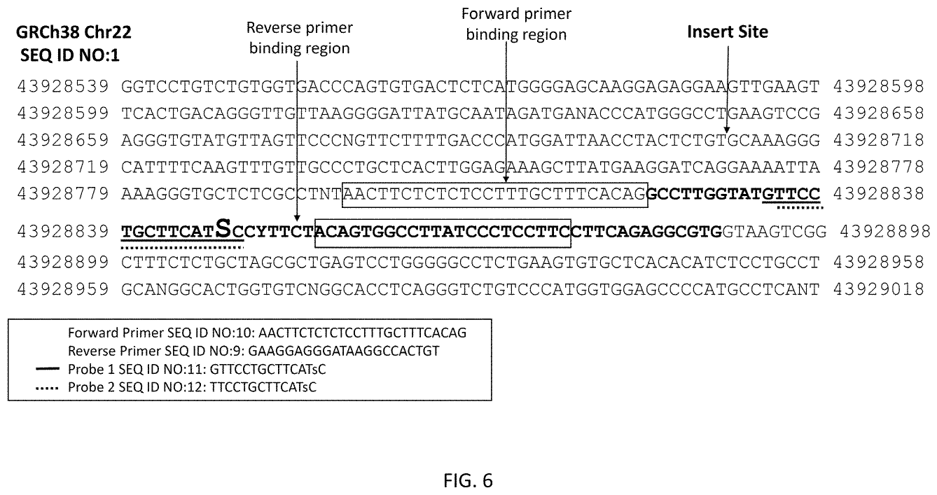

FIG. 6 depicts a custom assay design (primer/probe set 4) of a PNPLA3 I148M genotyping assay in accordance with embodiments of the disclosure. The length and position of the primers and probes is provided in TABLE 6. The forward primer is 26 bp long, starting at position 80. The reverse primer is 22 bp long, starting at position 158. Probe 1 is specific for the G allele of SNP rs738409. Probe 1 is 15 bp long, starting at position 116. Probe 2 is specific for the C allele of rs738409. Probe 2 is 14 bp long, starting at position 117.

TABLE 6
Primer/probe set 5

| Fwd | Fwd | Fwd | Fwd | Rev | Rev | Rev | Rev |
|---|---|---|---|---|---|---|---|
| Start | Length | Tm | % GC | Start | Length | Tm | % GC |
| 80 | 26 | 59.6 | 42 | 158 | 22 | 59.4 | 55 |

| Probe1 | Probe1 | Probe1 | Probe1 | Probe2 | Probe2 | Probe2 | Probe2 | Amp | |
|---|---|---|---|---|---|---|---|---|---|
| Start | Length | Tm | % GC | Start | Length | Tm | % GC | Len | Penalty |
| 116 | 15 | 67 | 53 | 117 | 14 | 66 | 50 | 79 | |

In certain embodiments, oligonucleotide probes (MGB probes) used in accordance with and/or provided by the present disclosure comprise one or more detectable entities or moieties, i.e., such molecules are “labeled” with such entities or moieties. In some embodiments, the detectable moiety is a visually detectable moiety.

Any of a wide variety of detectable agents can be used in the practice of the present invention. Suitable detectable agents include, but are not limited to: various ligands, radionuclides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

In some embodiments, the detectable moiety is biotin. Biotin can be bound to avidins (such as streptavidin), which are typically conjugated (directly or indirectly) to other moieties (e.g., fluorescent moieties) that are detectable themselves.

Below are described some non-limiting examples of other detectable moieties.

In some embodiments, detectable moiety is a visually detectable moiety. In further embodiments, the visually detectable moiety is a fluorescent dye. In some embodiments, the method comprises measuring the change in fluorescence of at least one dye associated with at least one probe. In other embodiments, the method comprises measuring the fluorescence of at least two dyes associated with at least two probes.

In certain embodiments, a detectable moiety is a fluorescent dye. Numerous known fluorescent dyes of a wide variety of chemical structures and physical characteristics are suitable for use in the practice of the present invention. A fluorescent detectable moiety can be stimulated by a laser with the emitted light captured by a detector. The detector can be a charge-coupled device (CCD) or a confocal microscope, which record its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED™, SPECTRUM GREEN™, cyanine dyes (e.g., CY-3™, CY-5™, CY-3.5™, CY5.5™, etc.), ALEXA FLUOR™ dyes (e.g., ALEXA FLUOR™ 350, ALEXA FLUOR™ 488, ALEXA FLUOR™ 532, ALEXA FLUOR™ 546, ALEXA FLUOR™ 568, ALEXA FLUOR™ 594, ALEXA FLUOR™ 633, ALEXA FLUOR™ 660, ALEXA FLUOR™ 680, etc.), BODIPY™ dyes (e.g., BODIPY™ FL, BODIPY™ R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™ 630/650, BODIPY™ 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as a fluorescent reporter dye. In certain embodiments, the selected reporter dyes are optimized to work together with minimal spectral overlap. Suitable reporter dyes (i.e., visually detectable moieties) include 6-carboxyfluorescein (FAM®), tetrachloro-6-carboxyfluorescein (TET), 2′-chloro-7′-phenyl-1,4-dichloro-6-carboxyfluorescein (VIC®), and the like.

In specific embodiments, a wild-type probe is labeled with a first fluorescent dye (e.g., FAM®) and a mutant probe is labeled with a second fluorescent dye (e.g., VIC®). Thus, a target sequence with homozygosity for allele C at the rs738409 loci will only produce a fluorescent signal from the first dye. A target sequence with heterozygosity (C/G) at the rs738409 loci will produce a fluorescent signal from the first dye and the second dye. A target sequence with homozygosity for allele G at the rs738409 loci will only produce a fluorescent signal from the second dye.

Additionally, in certain embodiments, the MGB probe comprises a NFQ. The NFQ is capable of absorbing signal from a fluorescent dye label (e.g., VIC and FAM). In some embodiments, the NFQ decreases background signal, thus, allowing for increased precision and sensitivity of the methods described herein. The NFQ is important for improving signal-to-noise ratio, and increasing assay sensitivity.

In certain embodiments, the oligonucleotide probe further comprises a nucleic acid specific for an allele of the SNP rs738409. Thus, in some embodiments, probes overlap with SNP rs738409. In further embodiments, the allele discrimination is located at the 3′ ends of the probes. Positioning the allele discrimination site at the 3′ end of the probe is important for increasing the binding specificity of the probes. In order to increase the specificity of the assay, in further embodiments, the probes do not overlap with SNP rs738408, which is located 3 bp downstream of the focal SNP, SNP rs738409.

In some embodiments, probe molecules that hybridize to the mutant or wild-type sequences can be used for detecting such sequences in the amplified product by solution phase or solid phase hybridization. Solid phase hybridization can be achieved, for example, by attaching the PNPLA3 probes to a microchip.

### PCR Amplification

In some embodiments, nucleic acids are amplified using polymerase chain reaction (PCR). Quantitative PCR (qPCR) or real-time PCR may be used for sensitive, specific detection and quantification of nucleic acid targets. Real-time PCR allows observation of genotyping data over time to evaluate the accuracy of genotype calls by observing the location of a given sample relative to others throughout all cycles. Thus, in some embodiments, the method further comprises PCR amplification using real-time PCR. In certain instances, the method further comprises multiplex PCR, in which several amplicons are amplified at once using multiple sets of primer pairs, may be employed. Amplification products can be examined by methods known in the art, including by visualizing (e.g., with one or more dyes).

The genomic sequence of the PNPLA3 gene and variant genotype and frequency information was obtained from the Ensembl database (www.ensembl.org). DNA sequence flanking the focal SNP rs738409 is shown in FIG. 1. In some embodiments, probes do not overlap with SNP rs738408. SNPrs738408 is 3 bp downstream of SNP rs73409. In further embodiments, the methods disclosed herein use short amplicon designs. Short amplicon designs increase robustness against varying specimen quality.

In certain instances, PCR amplification comprises using at least one primer pair to detect a specific SNP target. In specific embodiments, the primer pair comprises a forward primer and a reverse primer. In some embodiments, the primer pair is specific for a target nucleic acid sequence. In some instances, the primer/probe set comprises: (a) providing at least one oligonucleotide probe, wherein the probe comprises a nucleic acid specific for an allele of the SNP rs738409 and (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409. In some embodiments, the forward primer is fewer than 30, 25, 20, 15, 10, or 5 base pairs. In further embodiments, the forward primer is located at least 1, 5, 10, 15, 20, 25, 30, 40, 50, 60, 70, 80, 90, 100, 125, 150 base pairs downstream of a variant upstream of the SNP rs738409. In some embodiments, the reverse primer is fewer than 30, 25, 20, 15, 10, or 5 base pairs. In further embodiments, the reverse primer is located at least 1, 5, 10, 15, 20, 25, 30, 40, 50, 60, 70, 80, 90, 100, 125, 150 base pairs downstream of the SNP rs738409. For example, in some embodiments, the primer pair comprises: (a) a forward primer comprising any one of SEQ ID NOs: 2, 7, and 10 or any active fragment thereof, and (b) a reverse primer comprising any one of SEQ ID NOs: 3, 6, 8, and 9 or any active fragment thereof. In an embodiment, primer binding regions do not contain validated SNPs with population frequency >0.1%. In some embodiments, the forward and reverse primers are positioned such that the amplicon is less than 500, 400, 300, 250, 200, 250, 200, 150, 100, 50, or 25 base pairs.

In some embodiments, the probe has a higher annealing temperature than the PCR primers. Thus, in some instances, the probe binds the target sequence before the primers bind the target sequence.

In some embodiments, fluorescence measurements are collected during a post-PCR plate read. In further embodiments, these measurements may be used to plot the reporter signal normalized to the fluorescence signal (Rn) for each sample well. This data can be used to determine the genotype of the target nucleic acid present in the DNA sample.

In some embodiments, allelic discrimination plots may be used to analyze data. The allelic discrimination plots represent each sample well as an individual point on the plot. For example, an allelic discrimination plot may show clusters for sample wells containing genomic DNA homozygous for a first allele (wild-type), heterozygous, and homozygous for a second allele (mutant). Thus, in certain embodiment, an allelic discrimination plot may be used to determine the genotype of a sample containing a nucleic acid.

In some embodiments, the method further comprises primer extension. As the Taq polymerase extends the primer and synthesizes the nascent strand, the 5′ to 3′ exonuclease activity of the Taq polymerase degrades the probe that has annealed to the template. For example, during the primer extension phase of a PCR reaction, the probe is cleaved by 5′ to 3′ exonuclease activity of Taq polymerase. In some embodiments, probe cleavage dissociates the fluorophore from the quencher, and the resulting probe/allele specific fluorescence signal permits discrimination of homozygosity for allele C (fluorescence from dye 1 only), homozygosity for allele G (fluorescence from dye 2 only) and heterozygosity for allele C and allele G (fluorescence signals from both dyes) wherein the C allele (allele 1) binds the probe labeled with VIC and G allele (allele 2) binds the probe labeled with FAM. Thus, in an embodiment, an allelic discrimination plot would show a first cluster of samples homozygous for allele C, a second cluster heterozygous for allele C and allele G, and a third cluster of sample homozygous for allele G at the rs738409 loci of the PNPLA3 gene. In certain instances, the method measures the change in fluorescence of two dyes associated with the probes.

In some embodiments, the rs738409 PNPLA3 gene mutation is detected using an allele-specific amplification assay. In some embodiments of the PCR-based amplification methods, amplification primers may be used. Amplification primers can distinguish between different alleles (e.g., between a wild-type allele and a mutant allele).

In some embodiments, two complementary reactions are used. For example, one reaction may employ a primer specific for the wild type allele (“wild-type-specific reaction”) and the other reaction may employ a primer for the mutant allele (“mutant-specific reaction”). The two reactions may employ a common second primer. PCR primers specific for a particular allele (e.g., the wild-type allele or mutant allele) generally perfectly match one allelic variant of the target, but are mismatched to other allelic variant (e.g., the mutant allele or wild-type allele). The mismatch may be located at/near the 3′ end of the primer, leading to preferential amplification of the perfectly matched allele. Whether an amplification product can be detected from one or in both reactions indicates the absence or presence of the mutant allele. Detection of an amplification product only from the wild-type-specific reaction indicates presence of the wild-type allele only (e.g., homozygosity of the wild-type allele). Detection of an amplification product in the mutant-specific reaction only indicates presence of the mutant allele only (e.g. homozygosity of the mutant allele). Detection of amplification products from both reactions indicate (e.g., a heterozygote).

In some embodiments, it may be of interest to determine the presence or absence of additional allelic variants in the PNPLA3 gene. As discussed above, the PNPLA3 gene may comprise an insertion in the intron immediately upstream of exon 3. Thus, also disclosed is a method for detecting a genomic insertion linked to exon 3 of the PNPLA3 gene in a subject comprising: isolating nucleic acid (e.g., genomic DNA) from the subject; and amplifying by PCR at least a portion of the insertion sequence in the isolated DNA using a first primer that binds to the insertion sequence or a region upstream of the insertion sequence, and a second primer that binds to the PNPLA3 gene. In an embodiment, the first primer binds to the insertion sequence. In some embodiments, the insertion sequence comprises SEQ ID NO: 13, or the reverse complement of SEQ ID NO: 13. In some embodiments, the method may further comprise determining the nucleic acid sequence of the amplified product. For example, in some embodiments, the method may comprise determining that the insertion sequence comprises an allelic variation different from a wild-type sequence. In some embodiments, the first primer comprises or is the sequence of SEQ ID NO: 14. In some embodiments, the second primer comprises or is the sequence of SEQ ID NO: 15.

In certain embodiments, the method may further comprise determining if the allelic variant present in the insertion sequence is genetically linked to a particular allele at the rs738409 SNP. Thus, in certain embodiments, the method may comprise determining a genotype of exon 3 of the PNPLA3 gene. In some embodiments, determining the genotype comprises determining the sequence of a single nucleotide polymorphism (SNP) rs738409 of the PNPLA3 gene. For example, the method may comprise the steps of: (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote. The method may, in certain embodiments, further comprise determining if the allelic variant present in the insertion sequence is genetically linked to a particular allele at the rs738409 SNP.

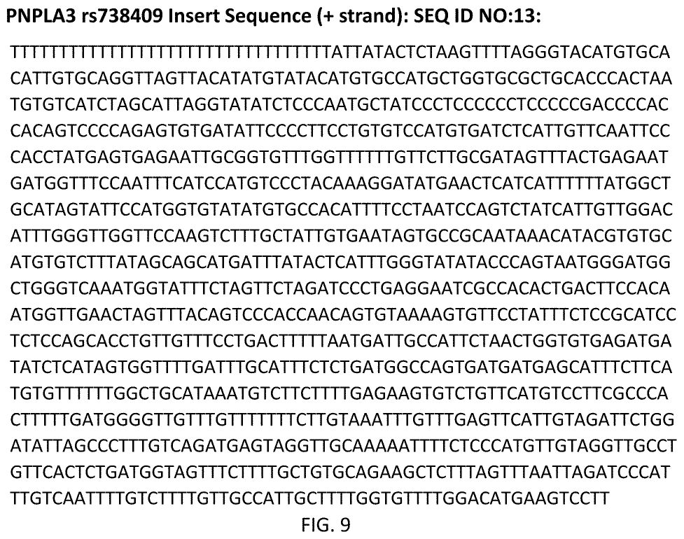

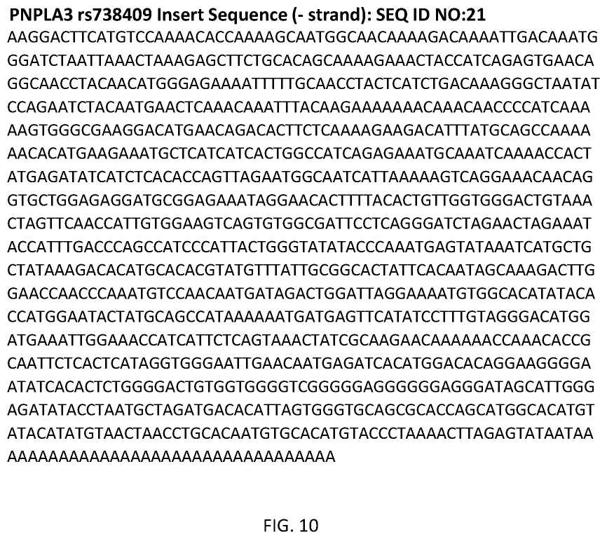

In some embodiments, the methods, compositions, and systems are capable of detecting the SNP rs738409 in the presence or absence of a variant in the region surrounding the PNPLA3 SNP rs738409 (SEQ ID NO. 1). Use of standard PCR sequencing methods may produce incorrect, non-specific calls for individuals who have an interfering variant. In some embodiments, the variant is an insertion. In certain embodiments, the insertion is at least 200, 250, 300, 250, 400, 450, 500, 750, 1000, 1500, 2000, 2500, 3000, 4000, or 5000 base pairs. In some embodiments, the variant is upstream or downstream of the SNP rs738409 linked to exon 3 of the PNPLA3 gene. FIG. 9 provides the positive strand sequence of an exemplary interfering insertion (SEQ ID NO:13). Thus, in some embodiments, the insertion comprises SEQ ID NO:13. FIG. 10 depicts the complementary, negative strand sequence (SEQ ID NO:21) of SEQ ID NO:13. FIG. 1 shows the position of the novel insertion variant is shown by the arrow. In some embodiments, the methods, compositions, and systems are capable of detecting the SNP rs738409 in the presence or absence of an insertion comprising SEQ ID NO:13 at the novel insertion site depicted in FIG. 1.

Thus, disclosed is a multiplexed, end-point, TaqMan allelic discrimination assay for the detection of PNPLA3 I148M. In certain instances, the assay includes one set of gene-specific primers paired with two allele-specific probes to detect the two possible variants of a single nucleic polymorphism (SNP) site in the targeted PNPLA3 gene sequence. The assay measures the change in fluorescence of the two dyes associated with the probes. In certain embodiments, the probe is designed to have a higher annealing temperature than the PCR primers such that the probe binds the target sequence earlier than the primers. During the primer extension phase of the PCR reaction, the probe is cleaved by 5′ to 3′ exonuclease activity of Taq polymerase. Probe cleavage dissociates the fluorophore from the quencher, and the resulting probe/allele specific fluorescence signal permits discrimination of homozygosity for allele C (fluorescence from dye 1 only), homozygosity for allele G (fluorescence from dye 2 only) and heterozygosity allele C and allele G (fluorescence signals from both dyes).

For example, the assay may use TaqMan MGB (minor groove binder) probes which incorporate a 5′ fluorescent reporter, a 3′ nonfluorescent quencher (NFQ) and a 3′ minor groove binder moiety. The MGB moiety increases the melting temperature (Tm) of the probe to shorten the probe length, stabilize probe binding and provide better sequence discrimination. The NFQ quenches signal from the fluorescent dye, combined with the short length of the MGB probe, they help lower background signal and increase assay sensitivity and precision. assay contains one allelic discrimination assay and 3 cell line gDNA controls with C/C, G/G and C/G genotype at the target locus, respectively.

### Compositions and Kits

In certain embodiments, the present disclosure provides compositions and/or kits for performing the disclosed methods. Generally, the compositions and/or kits described herein comprise one or more reagents that differentiate a normal rs738409 PNPLA3 gene or protein from a mutant rs738409 PNPLA3 gene or protein. For example, compositions and/or kits may comprise one or more (e.g., any combination of) reagents as described herein, and optionally additional components. In some embodiments, the individual components of the kit may be packaged together in a container.

In some embodiments, the composition and/or kit comprises at least one primer probe/set for detecting a SNP rs738409 of a PNPLA3 gene in a subject, wherein the primer/probe set comprises: (a) a mutant probe having a sequence of any one of SEQ ID NOs: 4, 11, 17, and 19; (b) a wild-type probe having a sequence of any one of SEQ ID NOs: 5, 12, 18, and 20; (c) a forward primer comprising a sequence of any one of SEQ ID NOs: 2, 7, and 10 or any active fragment thereof; and (d) a reverse primer comprising a sequence of any one of SEQ ID NOs: 3, 6, 8, and 9 or any active fragment thereof. In certain embodiments, the probe or probes comprise a MGB. Additionally and/or alternatively, the probes may be labeled with a detectable moiety and/or a quencher as disclosed herein.

Suitable reagents may include nucleic acid probes. In some embodiments, suitable reagents are provided in a form of an array such as a microarray or a PNPLA3 mutation panel.

For example, compositions and/or kits according to the invention may optionally contain buffers, enzymes, and/or reagents for use in methods described herein, e.g., for amplifying nucleic acids via primer-directed amplification. In certain instances, the composition and/or kit further comprises PNPLA3 I148M genotyping assay mix.

In some embodiments, the compositions and/or kits further comprise a control indicative of a healthy individual, e.g., a nucleic acid and/or protein sample from an individual who does not carry a mutant rs738409 PNPLA3 gene. In some embodiments, provided compositions and/or kits further comprise a control indicative of known rs738409 PNPLA3 C>G variant. Thus, in some embodiments, the composition and/or kit further comprises positive controls, such as but not limited to a C/C positive control, a C/G positive control, and a G/G positive control.

In some embodiments, the kits may also contain instructions on how to determine if an individual has NASH or a related disorder, is at risk of developing NASH or a NASH related disorder, or is a carrier of NASH mutation.

Also disclosed are compositions (e.g., kits) to characterize the presence and/or genotype of the insertion sequence or other variants that are upstream or downstream of the region of the human PNPLA3 gene that includes the rs738409 SNP. For example, disclosed is a composition and/or kit for detecting a genomic insertion linked to exon 3 of the PNPLA3 gene in a subject comprising: a first primer that binds to the insertion sequence or a region upstream of the insertion sequence, and a second primer that binds to the PNPLA3 gene. In an embodiment, the first primer binds to the insertion sequence. In certain embodiments, the insertion sequence comprises SEQ ID NO: 13, or the reverse complement of SEQ ID NO: 13.

In some embodiments, provided is a computer readable medium encoding instructions for performing any of the steps of any of the disclosed methods and/or for using any of the disclosed compositions and/or kits for determining the presence or absence of the rs738409 PNPLA3 C>G variant in a subject. For example, in certain embodiments, the computer readable medium comprises instructions for performing at least one of the steps of: (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote. Such computer readable medium may be included in a kit of the invention.

### Systems

In some embodiments, provided are systems for performing the methods disclosed herein and/or using any of the compositions and/or kits disclosed herein. Such systems may include a computer readable medium encoding instructions for performing any of the steps of any of the disclosed methods and/or for using any of the disclosed compositions and/or kits for determining the presence or absence of the rs738409 PNPLA3 C>G variant in a subject.

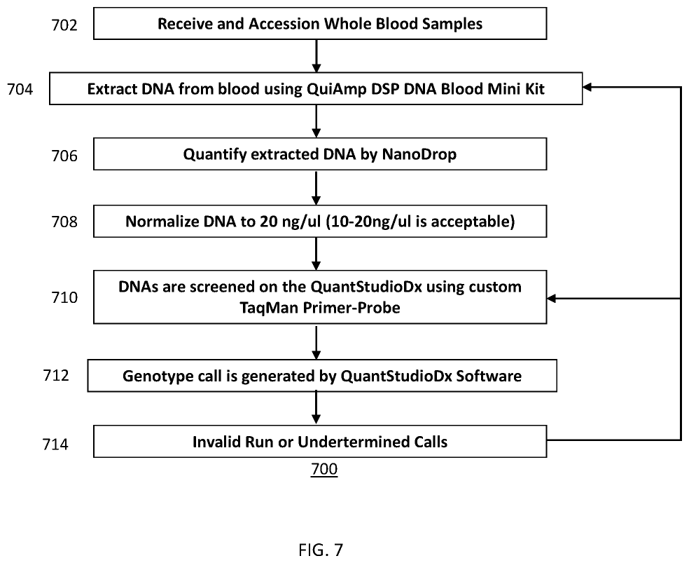

An example of such a system 700 is shown in FIG. 7. Thus, as disclosed in FIG. 7 the system may comprise a station or component (i.e., equipment and/or reagents) to receive samples 702. The system may also include a station or component to extract DNA from the samples 706. In an embodiment, a commercial kit, such as but not limited to a QiaAmp DSP DNA Blood Mini Kit may be used for DNA extraction. In certain embodiments, the system may further comprise a station or component for normalizing DNA 708. The system may also include a station and/or components for performing real time PCR detection of the rs738409 genotype 712. The system may also include a station for data analysis. For example, software such as the QuantStudioDX software may be used. Or, other computer-implemented software may be used. The data analysis may result in a visual representation of the data, for example, an allelic discrimination plot. In some cases, where an assignment cannot be made, the sample may be assayed again 714.

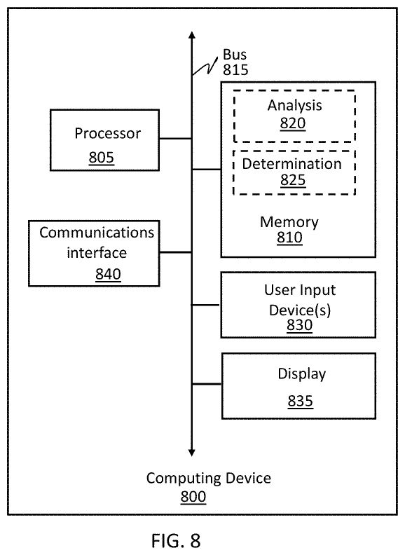

In an embodiment, any one of the steps may be controlled by a computing device 800 (FIG. 8). The computing device may comprise memory 810, a data analysis system 820 with software for determination of genotypes 825. The computing device 800, in this example, also includes one or more user input devices 830, such as a keyboard, mouse, touchscreen, microphone, etc., to accept user input. The computing device 800 also includes a display 835 to provide visual output to a user such as a user interface. The computing device 800 also includes a communications interface 840. In some examples, the communications interface 640 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### EXAMPLES

The following non-limiting examples has been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1. PNPLA3 I148M Genotyping Assay Method

For the validation and assay specificity studies, DNA was extracted from whole blood samples and quantified using NANODROP. The amount of DNA in each sample was normalized to 20 ng/μL.

The PNPLA3 assay mix was prepared by mixing two custom designed sequence-specific primers and two minor groove binder probes with non-fluorescent quenchers (MGBNFQ) (TABLE 7). One probe was labeled with VIC dye to detect the Allele 1 sequence; the second probe was labeled with FAM dye to detect the Allele 2 sequence for the targeted region of the PNPLA3 gene. A C/C, C/G, and G/G positive control and one negative control were used.

For PCR reactions, master mix was prepared in an amber tube to protect the assay probe from light according to TABLE 8. 8 μL of master mix was the aliquoted into wells of a MicroAmp Fast Optical 96-well plate. 2 μL of template or control were added to each reaction. The 96-well optical plate was sealed with an optical adhesive cover and spun down prior to proceeding with qPCR amplification on the QuantStudio DX platform The cycling conditions were set as shown in TABLE 9.

TABLE 7
PNPLA3 Assay Mix

| | | | | SEQ |
|---|---|---|---|---|
| Assay | Assay | | | ID |
| Target | ID | Oligo Type | Oligo Sequence | NO: |
| rs738409 | PNPLA3 | VIC Probe | TTCCTGCTTCATgCC | 17 |
| | | FAM Probe | CCTGCTTCATcCC | 18 |
| | | Forward | TTGCTTTCACAGGCC | 2 |
| | | Primer | TTGGT | |
| | | Reverse | GGAGGGATAAGGCCA | 3 |
| | | Primer | CTGTAGA | |

TABLE 8
Master Mix

| | | | Example for 8 |
|---|---|---|---|
| | 1 rxn | 1 rxn + | samples and 12 |
| Component | (ul) | 10% (ul) | controls: 20 rxns (ul) |
| PNPLA3 Assay Mix | 0.25 | 0.275 | 5.5 |
| TaqPath ProAmp Master | 5.0 | 5.5 | 100 |
| Mix | | | |
| Water | 2.75 | 3.025 | 60.5 |
| Total | 8 | NA | NA |

TABLE 9
Cycling Conditions

| Step | Temperature | Time | Cycles |
|---|---|---|---|
| Pre-Read | 60 C. | 30 sec | Hold |
| Initial denature/Enzyme activation | 95 C. | 5 min | |
| Denature | 95 C. | 5 sec | 40 |
| Anneal/Extend | 60 C. | 30 sec | |
| Post-Read | 60 C. | 30 sec | Hold |

### Validation of the PNPLA3 I148M Genotyping Assay

In total, 60 pre-characterized K₂EDTA whole blood specimens were used in the validation studies. In each study, an aliquot of blood specimens was extracted using IVD cleared and CE marked QIAamp DSP DNA Blood Mini Kit (Qiagen). Isolated DNA samples were then normalized to 20 ng/μL, or the concentration specified in the study, and 2 μL of template was used for each assay testing.

Six studies were conducted for this validation study. All studies met their respective acceptance criteria. Study objective, samples and acceptance criteria are summarized in Table 10. Study results are summarized in Table 11.

TABLE 10
Validation study design summary for PNPLA3 I148M Genotyping Assay

| Study Type | Objective | Samples | Acceptance Criteria |
|---|---|---|---|
| Precision | To evaluate between- | 6 Samples × 2 | All samples shall have |
| | day, between- | Replicates × 2 | point estimate of percent |
| | operator, between- | Instruments × 2 | agreement of 100% |
| | instrument and | Operators × 6 Days | across all categories. |
| | within-run assay | | |
| | reproducibility and | | |
| | repeatability. | | |
| Accuracy | The accuracy of the | Sixty (60) samples | Point estimate of percent |
| | PNPLA3 I148 | characterized as 10 | agreements from testing |
| | Genotyping Assay | G/G homozygotes, 25 | results between PNPLA3 |
| | was determined by | C/G heterozygotes and | I148M Genotyping |
| | comparing the results | 25 C/C homozygotes | Assay and the bi- |
| | of detection by Real- | | directional Sanger |
| | Time PCR versus | | sequencing shall be |
| | detection by Sanger | | 100% for each of the 3 |
| | sequencing. | | genotypes. |
| Assay | To determine the | 3 Samples × 5 Inputs × | For a given DNA input, |
| Robustness - | robustness of the | 12 Replicates | genotype concordance |
| DNA Input | assay by varying the | 5 Inputs: 80 ng, 60 ng, | shall be 100%, across all |
| | DNA input. | 40 ng, 20 ng and 1 ng | samples, replicates and |
| | | | lots. |
| | | | The robustness of the |
| | | | assay will be determined |
| | | | by the minimum DNA |
| | | | input that produces |
| | | | passing test result. |
| Specificity - No | To evaluate non- | All NTCs from the | Point estimate of percent |
| Template | specific signal | precision, robustness | of negative call shall be |
| Control | generation and | and accuracy studies | 100%. |
| | genotype calls. | (n = 84) | All NTCs shall be called |
| | | | “Negative Control |
| | | | (NC)” No AMPNC flag |
| | | | shall be observed for any |
| | | | NTCs in a valid run. |
| Assay | To evaluate the assay | PNPLA3 I148M | The BLAST analysis |
| Specificity - | probes and primers | Genotyping Assay | should not identify any |
| Alignment | for binding | Custom Primers and | off-target sequences. |
| Analysis | specificity. | Probes | |
| | | Sanger Sequencing | |
| | | Primers | |
| Specificity - | To identify any | PNPLA3 I148M | SNPCheck should not |
| SNPCheck | possible SNPs within | Genotyping Assay | identify validated |
| | the assay primer and | Custom Primers and | polymorphisms with |
| | probe binding | Probes | allele frequency >1% |
| | regions. | Sanger Sequencing | Only one target SNP |
| | | Primers | (rs738409) shall be |
| | | | identified from |
| | | | SNPcheck analysis on |
| | | | genotyping probe. |

TABLE 11
Validation results summary for PNPLA3 I148M Genotyping Assay

| Study Type | Experimental Design | Results |
|---|---|---|
| Precision | 6 Samples × 2 Replicates × 2 Instruments × | 100% Concordance |
| | 2 Operators × 6 Days = 288 tests | |
| Accuracy | Compare assay results with Sanger | 100% Concordance |
| | Sequencing results for 60 samples | |
| | 10 G/G homozygotes, 25 C/G | |
| | heterozygotes and 25 C/C homozygotes | |
| Assay Robustness - | 5 Inputs: 80 ng, 60 ng, 40 ng, 20 ng and 1 ng | 100% Concordance |
| DNA Input | 3 Samples × 5 Inputs × 12 Replicates = 180 | |
| | tests | |
| Specificity - No | All NTCs from the precision, robustness | 100% called as |
| Template Control | and accuracy studies included (n = 84) | “Negative Control” |
| | | No flag observed |
| Assay Specificity - | BLAST (in silico) analysis of custom | No off-target |
| Alignment Analysis | primers and probes | alignment |
| Specificity - | In silico analysis of custom primers and | Only target SNP |
| SNPCheck | probes for polymorphisms | identified |

### Example 2. Sanger Sequencing Assay

An experiment was performed using a standard Sanger sequencing design for a number of human specimens and the results demonstrated that routine design and Sanger analysis produced incorrect, non-specific calls for a number of human samples (TABLE 12). Conventional methods that are well known in the art for determining a single nucleotide polymorphism (SNP) include DNA analysis by Sanger sequencing. Typically, PCR primers are designed based on the known gene sequence to amplify a region of about 300-600 bases, wherein the SNP of interest would be included in the amplified DNA. The following PNPLA3 SNP rs738409 primers were designed: forward primer, GCCTGAAGTCCGAGGGTGT (SEQ ID NO:14), and reverse primer, TGTTGCCCTGCTCACTTGGAG (SEQ ID NO:16). The amplicon size from these standard designs would be predicted to be 288 base pairs. Standard PCR conditions well known in the art were used to amplify the DNA in this size range prior to Sanger sequencing. However, using standard design methods, if the PNPLA3 gene allele contained an insertion variant of sufficient size such as SEQ ID NO:13 (over one kilobase), the allele containing the insert was not amplified and thus did not yield DNA for Sanger sequencing results. This resulted in incorrect genotyping results as shown in TABLE 12.

TABLE 12
Results from Standard Sanger sequencing design
compared with the methods in this disclosure

| | | | Genotyping Result | |
|---|---|---|---|---|
| | Presence or Absence | | using primer design | |
| | of Insertion Variant | Sanger Genotyping | accounting for | |
| True | associated with | Result with routine | insertion variant | Count from 100 |
| Genotype | C allele | designed primers | (Example 1 method) | tested samples |
| CC | no variant | correct | correct | 10 |
| CC | homozygous | no call | correct | 17 |
| | variant | | | |
| CC | heterozygous | correct | correct | 24 |
| | variant | | | |
| CG | no variant | correct | correct | 17 |
| CG | variant with | inaccurate | correct | 22 |
| | C allele | genotype call | | |
| GG | no variant | correct | correct | 10 |

### Illustrations of Suitable Methods, Compositions, and Systems

Illustration A1 is a method for detection of a single nucleotide polymorphism (SNP) rs738409 of a Patatin-like phospholipase domain containing 3 (PNPLA3) gene in a subject comprising the steps of: (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote.

Illustration A2 is the method of any preceding or subsequent illustration, wherein the variant is an insertion.

Illustration A3 is the method of any preceding or subsequent illustration, wherein the variant is upstream or downstream of the SNP rs738409 linked to exon 3 of the PNPLA3 gene

Illustration A4 is the method of any preceding or subsequent illustration, wherein the variant comprises SEQ ID NO:13, or the reverse complement of SEQ ID NO: 13.

Illustration A5 is the method of any preceding or subsequent illustration, wherein the probe further comprises a minor grove binder moiety.

Illustration A6 is the method of any preceding or subsequent illustration, wherein the probe further comprises a visually detectable label.

Illustration A7 is the method of any preceding or subsequent illustration, wherein the probe further comprises a non-fluorescent quencher (NFQ).

Illustration A8 is the method of any preceding or subsequent illustration, wherein the at least one oligonucleotide probe is a mutant probe having a sequence of any one of SEQ ID NOs: 11, 17, and 19.

Illustration A9 is the method of any preceding or subsequent illustration, wherein the at least one oligonucleotide probe is a wild type probe having a sequence of any one of SEQ ID NOs: 5, 12, 18, and 20.

Illustration A10 is the method of any preceding or subsequent illustration, further comprising PCR amplification using at least one primer pair.

Illustration A11 is the method of any preceding or subsequent illustration, wherein the primer pair comprises: (a) a forward primer comprising any one of SEQ ID NOs: 2, 7, 10 or any active fragment thereof; and (b) a reverse primer comprising any one of SEQ ID NOs: 3, 6, 8, 9 or any active fragment thereof.

Illustration A12 is the method of any preceding or subsequent illustration, wherein the PCR amplification comprises a primer extension phase.

Illustration A13 is the method of any preceding or subsequent illustration, wherein the primer extension phase comprises cleavage of one or more probes by Taq polymerase such that the cleavage dissociates the visually detectable label from the quencher thereby resulting in a probe-allele specific visually detectable signal.

Illustration A14 is the method of any preceding or subsequent illustration, wherein the MGB moiety increases the melting temperature (Tm) of the probe.

Illustration A15 is the method of any preceding or subsequent illustration, wherein the visually detectable label is a fluorescent reporter.

Illustration A16 is the method of any preceding or subsequent illustration, wherein the probe binds to the nucleic acid from the subject before the primers during the PCR amplification.

Illustration A17 is the method of any preceding or subsequent illustration, wherein the genotype is used to identify a NASH patient who may benefit from a treatment.

Illustration Alb is the method of any preceding or subsequent illustration, further comprising treating NASH patients who are homozygotes (G/G) or heterozygotes (C/G) at the SNP rs738409 loci.

Illustration A19 is the method of any preceding or subsequent illustration, wherein the sample is obtained from cell-free DNA, cells, tissue serum, plasma, whole blood, urine, stool, saliva, buccal swabs, cord blood, chorionic villus sample, chorionic villus sample culture, amniotic fluid, amniotic fluid culture, transcervical lavage fluid, and any combination thereof.

Illustration A20 is the method of any preceding or subsequent illustration, wherein the nucleic acid from the subject is human genomic DNA.

Illustration A21 is the method of any preceding or subsequent illustration, further comprising detecting a genomic insertion linked to exon 3 of the PNPLA3 gene in a subject comprising amplifying by PCR at least a portion of the insertion sequence in the isolated DNA using a first primer that binds to the insertion sequence or a region upstream of the insertion sequence, and a second primer that binds to the PNPLA3 gene.

Illustration A22 is the method of any preceding or subsequent illustration, wherein the first primer binds to the insertion sequence.

Illustration A23 is the method of any preceding or subsequent illustration, wherein the insertion sequence comprises SEQ ID NO: 13, or the reverse complement of SEQ ID NO: 13.

Illustration B1 is a kit comprising at least one primer/probe set for detecting a SNP rs738409 of a PNPLA3 gene in a subject, wherein the primer/probe set comprises: (a) at least one oligonucleotide probe, wherein the probe comprises a nucleic acid specific for an allele of the SNP rs738409; and (b) a set of oligonucleotide PCR primers designed to amplify a region of the human PNPLA3 gene that includes the SNP rs738409 in the presence or absence of any portion of a variant in the human PNPLA3 gene, wherein the variant is upstream or downstream of the region of the human PNPLA3 gene that includes the SNP rs738409.

Illustration B2 is the kit of any preceding or subsequent illustration, wherein the primer/probe set comprises: (a) a mutant probe having a sequence comprising any one of SEQ ID NOs: 11, 17, and 19; (b) a wild-type probe having a sequence comprising any one of SEQ ID NOs: 12, 18, and 20; (c) a forward primer having a sequence comprising any one of SEQ ID NOs: 2, 7, 10 or any active fragment thereof; and (d) a reverse primer having a sequence comprising any one of SEQ ID NOs: 3, 6, 8, 9 or any active fragment thereof.

Illustration C1 is a system for performing the method or using the kit of any of the preceding or subsequent illustrations.

Illustration D1 is a method for detecting a genomic insertion linked to exon 3 of the PNPLA3 gene in a subject comprising: (a) isolating a nucleic acid from the subject; and (b) amplifying by PCR at least a portion of the insertion sequence in the isolated DNA using a first primer that binds to the insertion sequence or a region upstream of the insertion sequence, and a second primer that binds to the PNPLA3 gene.

Illustration D2 is the method of any preceding or subsequent illustration, wherein the first primer binds to the insertion sequence.

Illustration D3 is the method of any preceding or subsequent illustration, wherein the insertion sequence comprises SEQ ID NO: 13, or the reverse complement of SEQ ID NO: 13.

Illustration D4 is the method of any preceding or subsequent illustration, further comprising determining the nucleic acid sequence of the amplified product.

Illustration D5 is the method of any preceding or subsequent illustration, further comprising determining that the insertion sequence comprises an allelic variation different from a wild-type sequence.

Illustration D6 is the method of any preceding or subsequent illustration, further comprising determining a genotype of exon 3 of the PNPLA3 gene.

Illustration D7 is the method of any preceding or subsequent illustration, wherein determining the genotype comprises determining the sequence of a single nucleotide polymorphism (SNP) rs738409 of the PNPLA3 gene.

Illustration D8 is the method of any preceding or subsequent illustration, wherein determining the sequence of the rs738409 SNP comprises the steps of: (a) providing at least one oligonucleotide probe, wherein the probe comprises a nucleic acid specific for an allele of the SNP rs738409; (b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409; (c) contacting a sample comprising a nucleic acid from the subject with the at least one probe of step (a) such that hybridization occurs between the probe and the nucleic acid from the subject; (d) detecting binding of the probe to the SNP rs738409 by quantitative polymerase chain reaction (qPCR) amplification using the PCR primers of step (b); and (e) determining the genotype of the subject at the SNP rs738409 loci as homozygous for C, homozygous for G, or a C/G heterozygote.

We claim:

1. A method for detection of a single nucleotide polymorphism (SNP) rs738409 of a Patatin-like phospholipase domain containing 3 (PNPLA3) gene in a subject comprising the steps of:
(a) providing at least one oligonucleotide probe, wherein the probe comprises a nucleic acid specific for an allele of the SNP rs738409;
(b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409;
(c) contacting a sample comprising a nucleic acid from the subject with the at least one probe of step (a) such that hybridization occurs between the probe and the nucleic acid from the subject;
(d) detecting binding of the probe to the SNP rs738409 by quantitative polymerase chain reaction (qPCR) amplification using the PCR primers of step (b); and
(e) determining the genotype of the subject at the SNP rs738409 loci as homozygous for C, homozygous for G, or a C/G heterozygote.

2. The method of claim 1, wherein the variant is an insertion.

3. The method of claim 1, wherein the variant is upstream or downstream of the SNP rs738409 linked to exon 3 of the PNPLA3 gene.

4. The method of claim 1, wherein the variant comprises SEQ ID NO:13, or the reverse complement of SEQ ID NO: 13.

5. The method of claim 1, wherein the probe further comprises a minor grove binder moiety.

6. The method of claim 1, wherein the probe further comprises a visually detectable label.

7. The method of any claim 1, wherein the probe further comprises a non-fluorescent quencher (NFQ).

8. The method of claim 1 wherein, the at least one oligonucleotide probe is a mutant probe having a sequence of any one of SEQ ID NOs: 4, 11, 17, and 19.

9. The method of claim 1 wherein, the at least one oligonucleotide probe is a wild type probe having a sequence of any one of SEQ ID NOs: 5, 12, 18, and 20.

10. The method of claim 1, further comprising PCR amplification using at least one primer pair.

11. The method of claim 10, wherein the primer pair comprises:
(a) a forward primer comprising any one of SEQ ID NOs: 2, 7, 10 or any active fragment thereof; and
(b) a reverse primer comprising any one of SEQ ID NOs: 3, 6, 8, 9 or any active fragment thereof.

12. The method of claim 10, wherein the PCR amplification comprises a primer extension phase. Combine with 13

13. The method of claim 12, wherein the primer extension phase comprises cleavage of one or more probes by Taq polymerase such that the cleavage dissociates the visually detectable label from the quencher thereby resulting in a probe-allele specific visually detectable signal.

14. The method of claim 5, wherein the MGB moiety increases the melting temperature (Tm) of the probe.

15. The method of claim 6, wherein the visually detectable label is a fluorescent reporter.

16. The method of claim 10, wherein the probe binds to the nucleic acid from the subject before the primers during the PCR amplification.

17. The method of claim 1, wherein the genotype is used to identify a NASH patient who may benefit from a treatment.

18. The method of claim 17 further comprising treating NASH patients who are homozygotes (G/G) or heterozygotes (C/G) at the SNP rs738409 loci.

19. The method of claim 1, wherein the sample is obtained from cell-free DNA, cells, tissue serum, plasma, whole blood, urine, stool, saliva, buccal swabs, cord blood, chorionic villus sample, chorionic villus sample culture, amniotic fluid, amniotic fluid culture, transcervical lavage fluid, and any combination thereof.

20. The method of claim 1, wherein the nucleic acid from the subject is human genomic DNA.

21. The method of claim 1, further comprising detecting a genomic insertion linked to exon 3 of the PNPLA3 gene in a subject comprising amplifying by PCR at least a portion of the insertion sequence in the isolated DNA using a first primer that binds to the insertion sequence or a region upstream of the insertion sequence, and a second primer that binds to the PNPLA3 gene.

22. The method of claim 21, wherein the first primer binds to the insertion sequence.

23. The method of claim 21, wherein the insertion sequence comprises SEQ ID NO: 13, or the reverse complement of SEQ ID NO: 13.

24. A kit comprising at least one primer/probe set for detecting a SNP rs738409 of a PNPLA3 gene in a subject, wherein the primer/probe set comprises:
(a) at least one oligonucleotide probe, wherein the probe comprises a nucleic acid specific for an allele of the SNP rs738409; and
(b) a set of oligonucleotide PCR primers designed to amplify a region of the human PNPLA3 gene that includes the SNP rs738409 in the presence or absence of any portion of a variant in the human PNPLA3 gene, wherein the variant is upstream or downstream of the region of the human PNPLA3 gene that includes the SNP rs738409.

25. The kit of claim 24, wherein the primer/probe set comprises:
(a) a mutant probe having a sequence comprising any one of SEQ ID NOs: 4, 11, 17, and 19;
(b) a wild-type probe having a sequence comprising any one of SEQ ID NOs: 5, 12, 18, and 20;
(c) a forward primer having a sequence comprising any one of SEQ ID NOs: 2, 7, 10 or any active fragment thereof; and
(d) a reverse primer having a sequence comprising any one of SEQ ID NOs: 3, 6, 8, 9 or any active fragment thereof.

26. A system for performing the method or using the kit of any of the above claims.

27. A method for detecting a genomic insertion linked to exon 3 of the PNPLA3 gene in a subject comprising:
isolating a nucleic acid from the subject; and
amplifying by PCR at least a portion of the insertion sequence in the isolated DNA using a first primer that binds to the insertion sequence or a region upstream of the insertion sequence, and a second primer that binds to the PNPLA3 gene.

28. The method of claim 27, wherein the first primer binds to the insertion sequence.

29. The method of claim 27, wherein the insertion sequence comprises SEQ ID NO: 13, or the reverse complement of SEQ ID NO: 13.

30. The method of claim 27 further comprising determining the nucleic acid sequence of the amplified product.

31. The method of claim 27, further comprising determining that the insertion sequence comprises an allelic variation different from a wild-type sequence.

32. The method of claim 27, further comprising determining a genotype of exon 3 of the PNPLA3 gene.

33. The method of claim 32, wherein determining the genotype comprises determining the sequence of a single nucleotide polymorphism (SNP) rs738409 of the PNPLA3 gene.

34. The method of claim 33, wherein determining the sequence of the rs738409 SNP comprises the steps of:
(a) providing at least one oligonucleotide probe, wherein the probe comprises a nucleic acid specific for an allele of the SNP rs738409;
(b) providing a set of oligonucleotide PNPLA3-specific PCR primers designed to amplify an exon of the human PNPLA3 gene that includes the SNP rs738409 but not any variants that are upstream or downstream of the exon in the presence or absence of any portion of a variant in the human PNPLA3 gene, wherein the variant is upstream or downstream of the region of the human PNPLA3 gene that includes the SNP rs738409;
(c) contacting a sample comprising a nucleic acid from the subject with the at least one probe of step (a) such that hybridization occurs between the probe and the nucleic acid from the subject;
(d) detecting binding of the probe to the SNP rs738409 by quantitative polymerase chain reaction (qPCR) amplification using the PCR primers of step (b); and
(e) determining the genotype of the subject at the SNP rs738409 loci as homozygous for C, homozygous for G, or a C/G heterozygote.
